(19) United States
(12) Patent Application Publication
(10) Pub. No.: US 2025/0025717 A1
(43) Pub. Date: Jan. 23, 2025

(54) DEVICE FOR STIMULATING VITAMIN D PRODUCTION

(71) Applicant: Dyson Technology Limited, Malmesbury, Wiltshire (GB)

(72) Inventor: Benjamin John KNIGHT-GREGSON, Bristol (GB)

(21) Appl. No.: 18/716,547

(22) PCT Filed: Dec. 1, 2022
(86) PCT No.: PCT/GB2022/053045

§ 371 Date: Jun. 5, 2024

(30) Foreign Application Priority Data
Dec. 9, 2021 (GB) 2117790.2

(51) Int. Cl. A61N 5/06; A61M 37/00

(52) U.S. Cl. CPC A61N 5/0616; A61M 37/0015; A61N 2005/0661

(57) ABSTRACT

A device for stimulating vitamin D production in a tissue. The device comprises a UV-B light source configured to emit light with a wavelength between 280 nm and 320 nm; and at least one tissue penetrating member optically coupled to the UV-B light source to deliver light emitted from the UV-B light source into the tissue, wherein the at least one tissue penetrating member has a length greater than or equal to 10 μm and less than 10 mm.

### FIELD OF THE INVENTION

The present invention relates to a device for stimulating vitamin D production and particularly, although not exclusively, to a device for stimulating vitamin D production using a light source and at least one tissue penetrating member.

### BACKGROUND

Vitamin D is essential for human health. Vitamin D helps to maintain healthy bones and teeth, support the immune system, lung function and cardiovascular health, and protect against a range of diseases and conditions.

Generally, vitamin D can be obtained by humans through diet, for example by eating fatty fish, egg yolks, cheese, and mushrooms, for example. However, the most efficient way to help the body produce vitamin D is by exposure to sunlight.

Vitamin D is synthesized by the human body by vitamin D receptor cells in the skin that, through a chain of reactions, produce vitamin D (and in particular vitamin D₃) when they are exposed to ultraviolet B (UV-B) light from the sun.

Although vitamin D can be synthesized by the human body, a deficiency may still occur. For example, people who are not exposed to enough sunlight, e.g. those who live in northern latitudes, work nightshifts or in office environments, are homebound, or use a lot of sunscreen, may not produce sufficient vitamin D.

Furthermore, darker skin tones reduce the body's ability to absorb UV-B rays from the sun. As such, there is a strong correlation between skin pigmentation levels and vitamin D deficiency. For example, it is estimated that over 70% of African Americans are vitamin D deficient (Batai et al., 2021).

It is important to ensure that humans can obtain enough vitamin D to support their health.

The present invention has been devised in light of the above considerations.

### SUMMARY OF THE INVENTION

As a first aspect, there is disclosed a device for stimulating vitamin D production in a tissue, the device comprising:
a UV-B light source configured to emit light with a wavelength between 280 nm and 320 nm; and
at least one tissue penetrating member optically coupled to the UV-B light source to deliver light emitted from the UV-B light source into the tissue, wherein the at least one tissue penetrating member has a length greater than or equal to 10 μm and less than 10 mm.

In this way, when the device is positioned on tissue, such as human skin, UV-B light can travel through the at least one tissue penetrating member into the skin in order to stimulate vitamin D production. As the tissue penetrating member has a length greater than or equal to 10 μm but less than 10 mm, it can bypass the melanin layer of the skin and provide stimulus to the vitamin D producing cells underneath the melanin layer. Furthermore, as the melanin layer is bypassed, a higher efficiency of UV-B absorption is achieved by the vitamin D producing cells than if the UV-B source is delivered to the surface of the skin.

According, increased levels of vitamin D can be synthesized.

Optional features will now be set out. The following optional features are combinable singly or in combination with any aspect of the invention.

The tissue may be part of the human body, e.g. human skin for example.

As used herein, the term vitamin D may be understood as meaning vitamin D₃.

The UV-B light source may comprise one or more LEDs. It has been shown that vitamin D production via exposure to LED UV lighting is more efficient than that of sunlight (Kalajian et al., 2017). In particular, it was found that an LED can be 2.4 times more efficient in producing Vitamin D₃ in human skin that the sun in less than 1/60^{th} of the time. As such, by providing the UV-B light source as one or more LED, the efficiency of vitamin D production in the skin is increased. In other examples, an alternative UV-B light source may be used.

If the light source comprises a plurality of LEDs, the LEDs may emit light with the same or different wavelengths to each other.

As used herein, light having a wavelength at a defined value or within a defined range may be considered as light having a peak wavelength at the defined value or within the defined range.

As such, if the plurality of LEDs emit light with the same wavelength, they may be considered as emitting light having the same peak wavelength.

Preferably, the UV-B light source may be configured to emit light with a wavelength between 290 nm and 308 nm. It has been found that UV-B light within this wavelength range results in the most efficient vitamin D production in the skin.

The UV-B light source may be configured to emit light with a (peak) wavelength of 293 nm, 295 nm, 298 nm or 305 nm. For completeness, the numerical value of the wavelength as defined herein may be +/−0.5 nm.

It has been shown that using UV-B light with a wavelength at one or more of these particular values can generate increased levels of vitamin D in the skin (Kalajian et al., 2017).

The at least one tissue penetrating member may be a microneedle, for example.

As mentioned above, the at least one tissue penetrating member has a length greater than or equal to 10 μm and less than 10 mm. In this way, the at least one tissue penetrating member may extend into the tissue to a depth greater than, or equal to, approximately 10 μm, but less than 10 mm. The length of the at least one tissue penetrating member may be between 0.5 mm and 9 mm, more preferably between 0.5 mm and 4.5 mm, for example. The length of the at least one tissue penetrating member may be selected based on the intended position of the device on a human body.

In some examples, the device may comprise a plurality of tissue penetrating members, wherein each tissue penetrating member is optically coupled to the UV-B light source to deliver light emitted from the UV-B light source into the tissue. This increases the amount of UV-B light delivered to the skin.

The device may comprise at least 100, more preferably at least 200, more preferably at least 300, more preferably at least 400 tissue penetrating members.

Each tissue penetrating member may be optically coupled to a same UV-B LED. Alternatively, the tissue penetrating members may be coupled to different UV-B LEDs.

The plurality of tissue penetrating members may be arranged in an array. The array may be a 2D array. The array may be a square array, or a rectangular array. For example, the array may comprise 100 tissue penetrating members arranged in a 10×10 array. More preferably, the array may comprise 225 tissue penetrating members arranged in a 15×15 array. More preferably, the array may comprise 400 tissue penetrating members arranged in a 20×20 array.

In some examples, the device may comprise a substrate for supporting the at least one tissue penetrating member and the UV-B light source. The tissue penetrating member(s) and the UV-B light source may therefore be supported by a same substrate. In some examples, the device may comprise a plurality of substrates; e.g. a first substrate for supporting the at least one tissue penetrating member, and a second substrate for supporting the UV-B light source. The first and second substrate may be attached, or reversibly attachable, to one another to (reversibly) couple the at least one tissue penetrating member to the UV-B light source.

The/each substrate may be a flexible substrate, e.g. to mould to the contours of the human body. Alternatively, the/each substrate may be a rigid substrate.

The/each substrate may comprise a patch. The/each patch may have an adhesive layer thereon for attaching the device to the tissue. The adhesive layer may optionally be for reversibly attaching the device to the tissue. As such, the device may be discrete, and can be used under clothing, for example.

The substrate(s) may be at least partly optically transparent. In particular, the substrate(s) may be at least partly optically transparent in the wavelength range matching that of the UV-B light emitted from the UV-B light source, for example between 280 nm and 320 nm. In this way, the light emitted from the UV-B light source may be transmitted through the substrate(s) to the at least one tissue penetrating member.

The device may comprise a strap for strapping the device to the tissue. For example, the device may be configured to be worn on the wrist, and thus the strap may be sized to be worn on the wrist.

The at least one tissue penetrating member may be reversibly couplable to the UV-B light source. As such, the UV-B light source may be reusable. The at least one tissue penetrating member may be disposable such that it can be removed from the UV-B light source after use. The substrate may also be reusable (e.g. so that the substrate/patch and the UV-B light source can be used with disposable tissue penetrating members).

The tissue penetrating member(s) may be reversibly couplable to the UV-B light source by a mechanical connection. They may be reversibly couplable by their surfaces having a sufficient threshold of stiction to maintain coupled unless a user overcomes this threshold of stiction by pulling the surfaces apart. For example, the first substrate supporting the at least one tissue penetrating member may be reversibly couplable to the second substrate supporting the UV-B light source by the surfaces of the substrates having a sufficient threshold of stiction as set out above.

In some examples, the tissue penetrating member(s) may be reversibly couplable to the UV-B light source by interlocking slots. For example, slots/recesses may be formed on the first substrate supporting the tissue penetrating member(s), with complimentary protrusions formed on the second substrate supporting the UV-B light source (or vice versa). The slots/recesses may interlock with the complimentary protrusions to reversibly couple the two substrates, and thus the UV-B light source and the tissue penetrating member(s), together.

In further examples, the device may comprise an occluding layer (e.g. a patch) having an adhesive portion. In use, when the device is positioned on tissue (such as human skin), the occluding layer may be positioned over the UV-B light source and the substrate(s) to cover the UV-B light source and the substrate(s), and the adhesive portion may attach the device to the tissue. The adhesive portion may surround the substrate(s) around a periphery thereof.

In some examples, the UV-B light source and the at least one tissue penetrating member may be integrally formed. As such they may be irreversibly attached to each other. In particular, the UV-B light source may be adhered to the at least one tissue penetrating member (e.g. during manufacture) by an adhesive, such as an epoxy layer. For example, the first substrate supporting the at least one tissue penetrating member may be adhered to the second substrate supporting the UV-B light source by an adhesive. The adhesive (e.g. epoxy layer) may have suitable optical properties to allow light from the UV-B light source to travel therethrough to the at least one tissue penetrating member (e.g. be optically transparent in the UV-B wavelength range).

The device may further comprise a power source to power the UV-B light source. The power source may be one or more batteries. The power source may be rechargeable. The power source may be supported by (and attached to) the substrate, for example.

The device may further comprise a timer configured to provide user feedback when an activation period of the UV-B light source meets a predefined time limit. As such a user may be warned when the predefined time limit has been reached. This may ensure that UV-B exposure levels are within safe limits or safety guidelines.

The feedback may be audible (e.g. via a speaker on the device). The feedback may be visual (e.g. via a feedback light source on the device). The feedback may be via an external device, such as a mobile device. The device may be configured to communicate with an external device (wirelessly or via a wired connection) such that the user feedback is provided via the external device. As such a user may be provided with user feedback, such as an alert, via a mobile device when an activation period of the UV-B light source meets a predefined time limit.

The device may further comprise a controller configured to control the supply of power to the UV-B light source from the power source, and/or to control the timer.

The device may also comprise one or more user input interfaces, such as a button. The user input interface may be used to control activation of the UV-B light source (e.g. to turn it on and off).

According to a second aspect of the invention, there is provided use of the device of the first aspect to stimulate vitamin D production in a human body.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a device for stimulating vitamin D production, positioned on skin; and

FIG. 2 is a schematic illustration of a device for stimulating vitamin D production.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

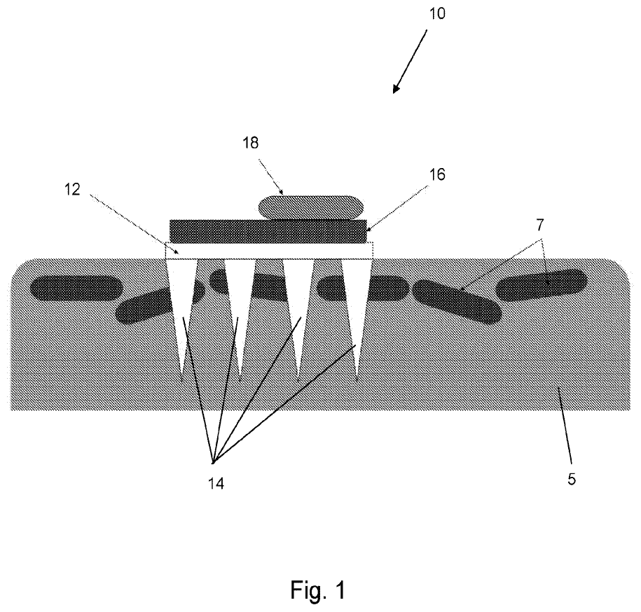

FIG. 1 shows a device 10 for stimulating vitamin D production. The device 10 is shown positioned on skin 5 of a user. The device 10 comprises a patch 12 which has an adhesive layer thereon so that the patch 12 can be attached to the skin 5. The patch 12 may be reversibly attachable to the skin 5, and may be flexible so that is can mould closely to the contours of the skin 5.

Extending, from one side of the patch 12 and in particular from the side of the patch configured to be attached to the skin 5 (by the adhesive layer), are a plurality of microneedles 14. As shown in FIG. 1, when the patch 12 is attached to the skin 2, the microneedles 14 extend into the skin 5, and past a melanin layer 7. Each microneedle 14 has a length greater than or equal to 10 μm, but less than 10 mm, and thus extends into the skin 5 to a depth greater than or equal to 10 μm but less than 10 mm. The microneedles 14 are optically transparent, and thus allow UV-B light to travel in a direction along a length of the microneedle and into the skin 5.

The device 10 also comprises a UV-B light source 16 on the opposite side of the patch 12 to the microneedles 14. In particular, the UV-B light source 16 may be attached to the patch 12 on the opposite side of the patch 12 to the microneedles 14. The UV-B light source 16 comprises a plurality of UV-B LEDs, each of which is optically coupled to a respective microneedle 14 such that the light emitted from the UV-B LEDs, through the patch 12 and microneedles 14, into the skin 5. The UV-B light can therefore be delivered to vitamin D producing cells in the skin 5, which can synthesize vitamin D.

The UV-B light source emits light having a peak wavelength between 280 and 320 nm.

The device 10 also comprises a battery 18 for providing power to the UV-B light source.

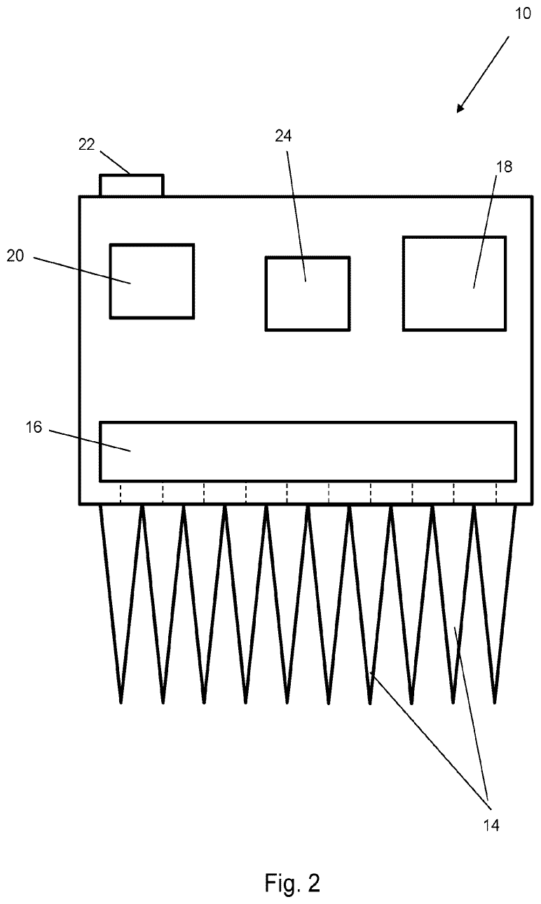

FIG. 2 is a schematic of device 10 for stimulating vitamin D production (not shown to scale). FIG. 2 shows how the plurality of microneedles 14, which are arranged in a 2D array, are optically coupled to the UV-B light source 16.

The device 10 also comprises a controller 20, which is powered by battery 18. Battery 18 supplies power to the UV-B light source 16 under the control of controller 20.

In particular, the controller 20 may control the supply of power to the UV-B light source 16 in response to a user input via a button 22. A user may be able to control the UV-B light source 16 to turn on and off by pressing the button 22. Of course, other user input devices may be used instead of a button 22.

The device 10 also comprises a timer 24. The timer 24 is configured to track an amount of time that the UV-B light source is switched on. A memory (not shown) of the device 10 is configured to store a predefined time limit, which may correspond to a maximum UV-B exposure level for a human. When the UV-B light source 16 is switched on, the timer 24 is configured to determine when an activation period (e.g. the amount of time that the UV-B light source is switched on) of the UV-B light source meets/exceeds the predefined time limit. When the predefined time limit is exceeded, the device 10 is configured to provide feedback to the user indicating that the predefined time limit has been met.

This feedback may be audible, visual and/or haptic feedback. For example, when the predefined time limit is met, a feedback light (e.g. LED) on the device may light up to indicate that the predefined time limit is met. In some examples, the feedback may be via an external device. In particular, the device 10 may communicate (e.g. wirelessly) with a mobile device, and the feedback may be provided to the user via the mobile device.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the invention and the state of the art to which the invention pertains. Full citations for these references are provided below. The entirety of each of these references is incorporated herein.
Batai K, Cui Z, Arora A, Shah-Williams E, Hernandez W, Ruden M, et al. (2021) Genetic loci associated with skin pigmentation in African Americans and their effects on vitamin D deficiency. PLOS Genet 17 (2): e1009319. https://doi.org/10.1371/journal.pgen.1009319
Kalajian, T. A., Aldoukhi, A., Veronikis, A. J. et al. Ultraviolet B Light Emitting Diodes (LEDs) Are More Efficient and Effective in Producing Vitamin D3 in Human Skin Compared to Natural Sunlight. Sci Rep 7, 11489 (2017). https://doi.org/10.1038/s41598-017-11362-2

1. A device for stimulating vitamin D production in a tissue, the device comprising:
a UV-B light source configured to emit light with a wavelength between 280 nm and 320 nm; and
at least one tissue penetrating member optically coupled to the UV-B light source to deliver light emitted from the UV-B light source into the tissue, wherein the at least one tissue penetrating member has a length greater than or equal to 10 μm and less than 10 mm.

2. The device according to claim 1, wherein the UV-B light source is an LED.

3. The device according to claim 1, wherein the UV-B light source is configured to emit light with a wavelength between 290 nm and 308 nm.

4. The device according to claim 1, wherein the UV-B light source is configured to emit light with a wavelength of 293 nm, 295 nm, 298 nm or 305 nm.

5. The device according to claim 1, wherein the at least one tissue penetrating member is a microneedle.

6. The device according to claim 1, wherein the device comprises a plurality of tissue penetrating members, wherein each tissue penetrating member is optically coupled to the UV-B light source to deliver light emitted from the UV-B light source into the tissue.

7. The device according to claim 6, wherein the plurality of tissue penetrating members are arranged in an array.

8. The device according to claim 1, wherein the device comprises a substrate for supporting the at least one tissue penetrating member and the UV-B light source.

9. The device according to claim 8, wherein the substrate comprises a patch with an adhesive layer thereon for reversibly attaching the device to the tissue.

10. The device according to claim 1, wherein the at least one tissue penetrating member is reversibly couplable to the UV-B light source.

11. The device according to claim 10, wherein the UV-B light source is reusable and the at least one tissue penetrating member is disposable.

12. The device according to claim 1, wherein the UV-B light source and the at least one tissue penetrating member are integrally formed.

13. The device according to claim 1, further comprising a battery to power the UV-B light source.

14. The device according to claim 1, further comprising a timer configured to provide user feedback when an activation period of the UV-B light source meets a predefined time limit.

15. Use of the device of claim 1 to stimulate vitamin D production in a human body.
